(19) United States
(12) Patent Application Publication
(10) Pub. No.: US 2023/0241286 A1
(43) Pub. Date: Aug. 3, 2023

(54) METHODS AND COMPOSITIONS FOR CATEGORIZING AND/OR TREATING CHIN RETRUSION IN A SUBJECT IN NEED THEREOF

(71) Applicant: Allergan, Inc., Irvine, CA (US)

(72) Inventors: Jiazhi Qu, Beijing (CN); Yi Jia, Beijing (CN); Dong Li, Beijing (CN)

(21) Appl. No.: 18/010,862

(22) PCT Filed: Jun. 17, 2021
(86) PCT No.: PCT/US2021/037966

§ 371 Date: Dec. 16, 2022

Related U.S. Application Data
(60) Provisional application No. 63/041,030, filed on Jun. 18, 2020.

(51) Int. Cl. A61L 27/20; G16H 50/20; G16H 50/70

(52) U.S. Cl. CPC A61L 27/20; G16H 50/20; G16H 50/70; A61L 2400/06

(57) ABSTRACT

The disclosure provides methods and compositions for treating or categorizing chin retrusion in a subject such as an East Asian subject. The disclosure also provides a chin retrusion scale useful for determining a degree of chin retrusion in a subject such as an East Asian subject.

### BACKGROUND

### Field of the Disclosure

The disclosure relates to methods and compositions for categorizing and/or treating chin retrusion in a subject in need thereof including, by categorizing a degree of chin retrusion in the subject.

### Description of the Related Art

A well-formed chin provides an essential foundation for an aesthetically pleasing face. Chin augmentation procedures, intended to enhance the contour of the chin and submental region appear to be gaining in popularity. (Bertossi D, Galzignato P F, Albanese M, Botti C, Botti G, Nocini P F, “Chin microgenia: a clinical comparative study,” Aesthetic Plast Surg. 2015; 39(5):651-658.) Indeed, the American Society for Aesthetic Plastic Surgery reported a 20% increase in these procedures from 2017 to 2018. (The American Society for Aesthetic Plastic Surgery's Cosmetic Surgery National Data Bank: Statistics 2018. Aesthet Surg J. 2019; 39(Suppl 4):1-27.) Further, chin augmentation was among the top 20 aesthetic procedures for both men and women and was most commonly performed among young adults.

A retrusive chin can be the result of changes in growth of the lower third of the face during maturation, trauma, or facial aging, the latter of which may exacerbate the deformities or asymmetries caused by the former two. The shape of the mandible affects the mouth, chin, and neck. As an individual ages, the reduction in skeletal support of this region makes soft tissue atrophy prominent, exaggerating jowls, decreasing chin protrusion, and making the jawline look weak. Chin deformities are among the most common bony abnormalities of the face, the most common of which is horizontal microgenia characterized by the presence of normal vertical height with a retruded bony chin.

Chin augmentation may be performed using surgical techniques, such as insertion of chin implants or genioplasty of osteotomy of the bony mentum, or less invasive procedures, such as injectable soft-tissue filler treatments, which have become increasingly popular. Individuals primarily choose to undergo cosmetic chin augmentation surgery to improve their emotional and social interactions and concerns about how others perceive them based on appearance. (See, e.g., Furnham A, Levitas J, “Factors that motivate people to undergo cosmetic surgery,” Can J Plast Surg. 2012; 20(4): e47-50; Moosavizadeh S M, Niazi F, Kalantar-Hormozi A, “Evaluation of female patients motivating factors for aesthetic surgery,” World J Plast Surg. 2012; 1(2):76-82; Nellis J C, Ishii M, Papel I D, et al., “Association of face-lift surgery with social perception, age, attractiveness, health, and success,” JAMA Facial Plast Surg. 2017; 19(4):311-317.)

Lack of alignment between the chin and lips (e.g., chin retrusion) is considered unattractive in many countries/regions. (See, e.g., Naini F B, Donaldson A N, McDonald F, Cobourne M T. Assessing the influence of chin prominence on perceived attractiveness in the orthognathic patient, clinician and layperson. Int J Oral Maxillofac Surg. 2012; 41(7):839-846.) A photonumeric scale often used to assess chin prominence in Western men and women uses anthropometric landmarks of the lower face to derive objective estimates of the degree of retrusion. (Sykes J M, Carruthers A, Hardas B, et al., “Development and validation of a photonumeric scale for assessment of chin retrusion,” Dermatol Surg. 2016; 42(suppl 1):5211-5218.)

Although valid and reliable for Western patients, however, art recognized methods are different for use in non-Caucasian subjects due to diverging craniofacial morphology characteristics, especially in the lower face, including a significantly different mandibular plane angle, and distinct aesthetic goals, including a different ideal mandible-lip ratio. Thus, a validated scale does not exist to objectively rate the existence of severe or extreme chin retrusion in non-Caucasian patient populations that may be amendable to therapeutic intervention. Dermatologists and plastic surgeons who treat men and women seeking aesthetic improvements would be well served by a validated scale that can be uniformly applied.

Thus, there exists a strong need for methods and compositions for identifying, classifying and treating severe or extreme chin retrusion in subjects. In addition, there exists a strong need for a validated scale useful for identifying, classifying and treating severe or extreme chin retrusion in subjects. Furthermore, there exists a strong need for methods of evaluating the degree of change in the severity or extremity of chin retrusion in subjects.

### SUMMARY

The present disclosure provides methods and compositions for treating chin retrusion in a subject in need thereof (e.g., a non-Caucasian or Asian subject). The methods may comprise generating an image of a face of the subject having a defined chin midpoint, a supramentale line, and a cheilion vertical line; determining a position of the defined chin midpoint relative to the supramentale line and the cheilion vertical line; and treating chin retrusion in the subject if the defined chin midpoint is at the supramentale line, between the supramentale line and the cheilion vertical line, or at or behind the cheilion vertical line. In certain embodiments, chin retrusion is treated if the defined chin midpoint is at or behind the cheilion vertical line. The position of the defined chin midpoint may be determined by a physician or a medical practitioner.

In some embodiments of each or any of the above- or below-mentioned embodiments, the subject is non-Caucasian. In certain embodiments, the subject is an East Asian subject.

In some embodiments of each or any of the above- or below-mentioned embodiments, the methods comprise administering a soft tissue filler composition comprising a hyaluronic acid (HA) component crosslinked with a crosslinking agent to the subject. In certain embodiments, the composition can comprise a HA component crosslinked using a crosslinking agent selected from 1,4-butanediol diglycidyl ether (or 1,4-bis(2,3-epoxypropoxy)butane, 1,2-bis(2,3-epoxypropoxy)ethylene, or 1-(2,3-epoxypropyl)-2,3-epoxycyclohexane.

In some embodiments of each or any of the above- or below-mentioned embodiments, the composition comprises HA in an amount greater than about 20 mg/ml.

In some embodiments of each or any of the above- or below-mentioned embodiments, the composition comprises high molecular weight HA and low molecular weight HA. In certain embodiments, the HA of the composition is a mixture containing at least about 50% by weight low molecular weight HA, based on a total weight of the HA comprising the composition.

In some embodiments of each or any of the above- or below-mentioned embodiments, the composition comprises HA crosslinked with a crosslinking agent, wherein the HA has a degree of crosslinking of between about 4% and about 10%. In certain embodiments, the HA has a degree of crosslinking of about 4%, about 5%, about 6%, about 6.5%, about 7%, about 8%, about 9%, or about 10%.

In some embodiments of each or any of the above- or below-mentioned embodiments, the composition comprises lidocaine combined with a crosslinked HA component. In further embodiments, lidocaine is present in the composition at a concentration of about 0.05% w/w to about 1% w/w.

In some embodiments of each or any of the above- or below-mentioned embodiments, the composition is administered subcutaneously and/or supraperiosteally into at least one treatment area of the face of the subject, wherein the treatment area is selected from the group consisting of the pogonion, the menton, the left pre jowl sulcus, the right pre jowl sulcus, and the sublabial (mental) crease.

The present disclosure also provides methods for categorizing a subject based on a degree of chin retrusion. Such methods may comprise generating an image of a face of the subject having a defined chin midpoint and one or more of a labrale inferius line, a supramentale line, and a cheilion vertical line; determining a position of the defined chin midpoint relative to the supramentale line and cheilion vertical line; and categorizing a degree of chin retrusion in the subject based on the position of the defined chin midpoint relative to the labrale inferius line, the supramentale line, or the cheilion vertical line.

In some embodiments of each or any of the above- or below-mentioned embodiments, the subject is categorized as having no chin retrusion if the defined chin midpoint of the subject is at or in front on the labrale inferius line of the subject. In certain embodiments, the subject is categorized as having minimal chin retrusion if the defined chin midpoint is between the labrale inferius line and the supramentale line. In further embodiments, the subject is categorized as having moderate chin retrusion if the defined chin midpoint is at the supramentale line.

In some embodiments of each or any of the above- or below-mentioned embodiments, the subject is categorized as having moderate-severe chin retrusion if the defined chin midpoint is between the supramentale line and the cheilion vertical line. In some embodiments, the subject is categorized as having severechin retrusion if the defined chin midpoint is at or behind the cheilion vertical line.

The present disclosure also provides a chin retrusion scale. In some embodiments, the chin retrusion scale can comprise a plurality of facial images representing a plurality of grades of chin retrusion, wherein each image comprises a profile view of a face showing at least a defined chin midpoint and one or more of a supramentale line, a labrale inferius line, and a cheilion vertical line. In further embodiments, the disclosure provides a photonumeric scale of chin retrusion. In still further embodiments, the photonumeric scale can comprise scores of 0 to 4 (e.g., 0, 1, 2, 3, and 4), wherein a score of 0 signifies no chin retrusion is present in a subject that exhibits a defined chin midpoint at or in front on the labrale inferius line; a score of 1 signifies minimal chin retrusion in a subject that exhibits a defined chin midpoint between the labrale inferius line and the supramentale line; a score of 2 signifies moderate chin retrusion in a subject that exhibits a defined chin midpoint at the supramentale line; a score of 3 signifies moderate-severe chin retrusion in a subject that exhibits a defined chin midpoint between the supramentale line and the cheilion vertical line; and a score of 4 signifies severe chin retrusion in a subject that exhibits a defined chin midpoint at or behind the cheilion vertical line.

The present disclosure also provides methods of treating chin retrusion in a subject in need thereof comprising, generating an image of the face of a subject having a defined chin midpoint and one or more of a supramentale line and a cheilion vertical line; and categorizing a degree of chin retrusion in the subject, wherein moderate chin retrusion is present if the defined chin midpoint is at the supramentale line, moderate-severe chin retrusion is present if the defined chin midpoint is between the supramentale line and the cheilion vertical line, and severechin retrusion is present if the defined chin midpoint is at or behind the cheilion vertical line; and treating chin retrusion if the subject has moderate, moderate-severe, or severe chin retrusion.

The present disclosure also provides methods for determining a change in a degree of chin retrusion in a subject. In some embodiments, the method comprises determining a change in degree of chin retrusion in a subject after a dermal filler is administered to the face of the subject. In further embodiments, the method comprises determining a change in degree of chin retrusion in a subject as the subject ages.

The disclosure also provides methods for determining a change in a degree of chin retrusion in a subject comprising generating a first image of a face of the subject having a defined chin midpoint, and two or more of a labrale inferius line, a supramentale line, and a cheilion vertical line; determining from the first image a first position of the defined chin midpoint relative to the labrale inferius line, supramentale line and/or the cheilion vertical line; and assigning a first degree of chin retrusion in the subject based on the first position of the defined chin midpoint relative to the labrale inferius line, the supramentale line and/or the cheilion vertical line; generating a second image of the face of the subject; determining from the second image a second position of the defined chin midpoint relative to the labrale inferius line, the supramentale line and/or the cheilion vertical line; and assigning a second degree of chin retrusion in the subject base on the second position of the defined chin midpoint relative to the labrale inferius line, the supramentale line, and/or the cheilion vertical line.

In some embodiments of each or any of the above- or below-mentioned embodiments, the methods further comprise administering a treatment to a treatment area on the face of the subject before the step of generating a second image. In certain embodiments, the treatment can comprise administering a soft tissue filler. In some embodiments, the second degree of chin retrusion is less than the first degree of chin retrusion. In further embodiments, the second image is generated at between about 1 month and about 24 months after the first image. In a further embodiment, the second image is generated about 36, 48, 60, 72, 84, 96, or more months after the first image.

The present disclosure also provides a computing platform for use by medical providers who treat patients seeking cosmetic procedures. In some embodiments, the platform stores and analyzes a plurality of images of faces having a defined chin midpoint, a labrale inferius line, a supramentale line, and a cheilion vertical line, or information associated with images of faces (including images with a categorized degree of chin retrusion), and uses machine learning and pattern recognition to categorize a degree of chin retrusion in a subject and optionally make treatment recommendations in order to (i) reduce errors for practitioners and (ii) achieve better outcomes for patients.

In some embodiments of each or any of the above- or below-mentioned embodiments, a method of providing safe and accurate treatment recommendations is implemented at a computer system having one or more processors and memory storing one or more programs for execution by the one or more processors. The method includes obtaining an input image of a face having a defined chin midpoint, and two or more of a labrale inferius line, a supramentale line, and/or a cheilion vertical line; comparing, using a pattern recognition process, one or more aspects of the input image to corresponding aspects of a plurality of reference images having a defined chin midpoint, a labrale inferius line, a supramentale line, and/or a cheilion vertical line; obtaining, based on a result of the comparing, categorizing a degree of chin retrusion in the subject (e.g., assigning the degree of chin retrusion a score as disclosed herein); and optionally recommending a treatment plan based on the input image, wherein the reference images are each associated with a chin retrusion score (e.g., score 0, 1, 2, 3, or 4 as disclosed herein).

In some embodiments of each or any of the above- or below-mentioned embodiments, the computer system includes memory storing instructions for causing the computer system to perform any of the methods described herein. In further embodiments, the instructions stored in memory of a computer system include instructions for causing the computer system to perform any of the methods described herein.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 shows an assessment guide indicating facial landmarks useful for practicing embodiments of the disclosure.

FIGS. 2A-2E show representative images in lateral profile view illustrating cases with no chin retrusion (Grade 0, FIG. 2A), minimal chin retrusion (Grade 1, FIG. 2B), moderate chin retrusion (Grade 2, FIG. 2C), moderate-severe chin retrusion (Grade 3, FIG. 2D), and severe chin retrusion (Grade 4, FIG. 2E) categorized according to methods of the disclosure.

FIG. 3 shows representative images demonstrating degrees of chin retrusion categorized according to methods of the disclosure.

FIG. 4 shows a flowchart for validation of a chin retrusion scale according to methods of the disclosure.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details.

The present disclosure provides methods and compositions for treating chin retrusion in a subject (e.g., a non-Caucasian or East Asian subject) in need thereof. The methods may comprise generating an image of a face of the subject having a defined chin midpoint, a supramentale line, and a cheilion vertical line; determining a position of the defined chin midpoint relative to the supramentale line and the cheilion vertical line; and treating chin retrusion in the subject if the defined chin midpoint is at the supramentale line, between the supramentale line and the cheilion vertical line, or at or behind the cheilion vertical line.

As used herein, a non-Caucasian, or non-Western, subject means a person who is not of European origin, and who therefore lacks the anthropometric landmarks of the lower face used in art-recognized methods of assessing chin retrusion in Caucasian subjects. For example, Caucasian and non-Caucasian subjects exhibit diverging craniofacial morphology characteristics, especially in the lower face, including a significantly different mandibular plane angle, and distinct aesthetic goals, including a different ideal mandible-lip ratio.

As used herein, “East Asian” refers to a subset of non-Caucasian subjects who are of East Asian descent or origin, for example, from China, Hong Kong, Macau, Taiwan, Japan, Mongolia, North Korea, or South Korea.

As used herein, “chin retrusion” describes an anatomical state characterized by a lack of adequate chin projection relative to other features of the face. Chin retrusion may be assessed by the methods disclosed herein. Other means of assessing chin retrusion are known in the art. For example, the angle defined by the glabella, subnasale, and pogonion (the “G-Sn-Pog, “POG,” or “Burstone” angle) may be used to characterize chin retrusion. In such an embodiment, a subject with a POG angle equal to or less than 172.5° is treated by the methods disclosed herein.

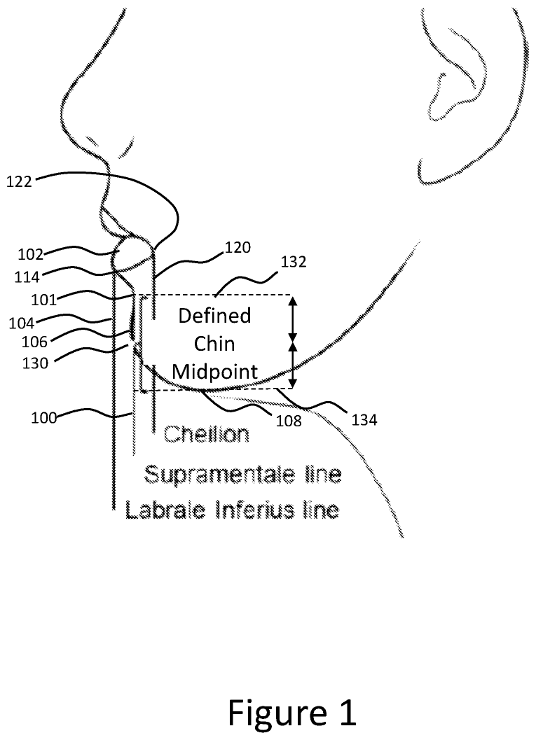

As used herein and illustrated in FIG. 1, a “supramentale line” 100 demarcates the point of greatest concavity, the “supramentale” 101 (also known as the labiomental sulcus), in the midline (e.g., along the midsagittal or median plane of the body) between the lower lip 102 and soft tissue pogonion 106. The soft tissue pogonion 106 is the most forward-projecting point on the anterior surface of the chin. Furthermore, as used herein and illustrated in FIG. 1, a “labrale inferious line” 104 means a line demarcating the most forward (anterior) projecting point in the midline of the lower vermilion border 114. The vermilion is the red portion of the lips, and the vermilion border 114 is the rim of skin demarcating the vermilion. As used herein and illustrated in FIG. 1, a “cheilion vertical line” 120 means a line demarcating the coronal plane that bisects the cheilion 122, the position where the lateral aspects of the vermilion of the upper and lower lips join.

As used herein and illustrated in FIG. 1, a “defined chin midpoint” 130 means the most anterior point on the midline of the chin (e.g., along the midsagittal or median plane of the chin), when the face is seen in lateral 90° view, equidistant from a first line 132 extending in a transverse or horizontal plane that bisects the supramentale 101 and a second, parallel line 134 extending in a transverse or horizontal plane that bisects the gnathion 108.

The step of generating an image of a face of a subject may be accomplished by methods known in the art. Such methods may comprise capturing a photographic image. For example, in some embodiments an image is captured by digital or conventional photography. In certain embodiments, capturing an image of the face of the subject can comprise photographing the subject's face, without smiling, from a left, approximately lateral, about 90° view in natural light, with a relaxed, natural posture and with tragus points exposed and the Frankfort horizontal plane parallel to the floor. In some embodiments, capturing an image of the face of the subject can comprise capturing a lateral image depicting at least a gnathion, a supramentale, a supramentale line, a cheilion, and a cheilion vertical line.

In some embodiments, methods of the disclosure further comprise determining a position of the defined chin midpoint relative to other facial landmarks. For example the position of the defined chin midpoint may be determined relative to the supramentale line and cheilion vertical line.

The step of determining a position of the defined chin midpoint may comprise determining the relative position of the defined chin midpoint by reference to other facial landmarks. For example, the position of the defined chin midpoint may be determined to be at or behind (i.e., posterior to) a facial landmark, such as the supramentale line and/or the cheilion vertical line. Alternatively, the position of the defined chin midpoint may be determined to be in front of (i.e., anterior to) a facial landmark. Still further, the position of the defined chin midpoint may be determined to be between two facial landmarks, such as between the supramentale line and the cheilion vertical line.

Additionally, the step of determining a position of the defined chin midpoint may comprise determining the specific distance of the defined chin midpoint relative to other facial landmarks. For example, the position of the defined chin midpoint may be determined by measuring a specific distance between the defined chin midpoint and one or more facial landmarks, such as the supramentale line and/or the cheilion vertical line.

The present disclosure also provides methods for categorizing a subject (e.g., a non-Caucasian or East Asian subject) based on their degree of chin retrusion. Such methods may comprise generating an image of a face of the subject comprising at least a defined chin midpoint, a labrale inferius line, a supramentale line, and a cheilion vertical line; determining the position of the defined chin midpoint relative to the supramentale line and cheilion vertical line; and categorizing a degree of chin retrusion in the subject based on the position of the defined chin midpoint relative to the supramentale line and cheilion vertical line.

The subject may be categorized as having no chin retrusion if the defined chin midpoint is at or in front of (anterior to) the labrale inferius line, as having minimal chin retrusion if the defined chin midpoint is between the labrale inferius line and the supramentale line, as having moderate chin retrusion if the defined chin midpoint is at the supramentale line, as having moderate-severe chin retrusion if the defined chin midpoint is between the supramentale line and the cheilion vertical line, or as having severechin retrusion if the defined chin midpoint is at or behind the cheilion vertical line.

The present disclosure also provides a scale for determining the degree of chin retrusion in a subject, or a “chin retrusion scale.” A chin retrusion scale may comprise a photonumeric scale comprising representative images of different categories of chin retrusion in subjects and descriptions for each category. For example, a photonumeric chin retrusion scale may comprise representative images corresponding to two or more of: no chin retrusion, minimal chin retrusion, moderate chin retrusion, moderate-severe chin retrusion, and severe chin retrusion.

The photonumeric chin retrusion scale may comprise scores from 0-4 (e.g., 0, 1, 2, 3, and 4), wherein: a score of 0 signifies no chin retrusion in a subject with a defined chin midpoint at or in front on the labrale inferius line; a score of 1 signifies minimal chin retrusion in a subject with a defined chin midpoint between the labrale inferius line and the supramentale line; a score of 2 signifies moderate chin retrusion in a subject with a defined chin midpoint at the supramentale line; a score of 3 signifies moderate-severe chin retrusion in a subject with a defined chin midpoint between the supramentale line and the cheilion vertical line; and a score of 4 signifies severe chin retrusion in a subject with a defined chin midpoint at or behind the cheilion vertical line.

The present disclosure also provides methods and compositions for treating chin retrusion in a subject in need thereof including, for example, increasing chin protrusion in the subject in need thereof. Such methods may include a range of surgical techniques, such as osteotomy of the bony mentum or insertion of soft-tissue allografts or alloplastic implants. Surgical methods of treatment include osseous genioplasty and alloplastic augmentation.

Additionally or alternatively, the methods may comprise administering an injectable soft-tissue filler composition.

A wide variety of injectable soft tissue fillers are known to the art, including, for example, compositions comprising hyaluronic acid, collagen, calcium hydroxylapatite, and/or poly-L-lactic acid. Additional dermal filler compositions comprise, for example, polymethylmethacrylate microspheres, hydrogel polymers, and/or silicone. Soft tissue fillers are suitable for a variety of cosmetic and therapeutic indications, including chin augmentation or correction in subjects in need thereof, such as subjects with chin retrusion. The composition may be used for injection into the subcutaneous and/or supraperiosteal space and preferably are moldable after injection, and therefore permit sculpting, contouring, and shaping across the injected areas, for example, the chin and jaw area.

In some embodiments, the methods of the disclosure comprise treating chin retrusion by administering a soft tissue filler composition comprising a hyaluronic acid (HA) component crosslinked with a crosslinking agent to the subject. As used herein, HA can refer to any of its hyaluronate salts, and includes, but is not limited to, sodium hyaluronate (NaHA), potassium hyaluronate, magnesium hyaluronate, calcium hyaluronate, and combinations thereof. Both HA and pharmaceutically acceptable salts thereof can be used in this disclosure.

The soft-tissue filler compositions may comprise HA crosslinked with a crosslinking agent selected from the group consisting of 1,4-butanediol diglycidyl ether (BDDE), 1,4-bis(2,3-epoxypropoxy)butane, 1,4-bisglycidyloxybutane, 1,2-bis(2,3-epoxypropoxy)ethylene and 1-(2,3-epoxypropyl)-2,3-epoxycyclohexanethe. Preferably, the injectable filler compositions generally comprise HA crosslinked with BDDE. Such compositions are suitable for injection, for example, through a fine gauge needle, and are capable of augmenting, correcting, or creating volume or lift in the face, for example, the lower face, for example, the chin or jaw, or for the midface, for example, the nose.

The soft-tissue filler compositions of the disclosure may comprise HA with a degree of crosslinking of between about 4% and about 12%. “Degree of crosslinking” as used herein refers to the intermolecular junctions joining the individual HA polymer molecules, or monomer chains, into a permanent structure, or as disclosed herein the soft tissue filler composition. Moreover, degree of crosslinking for purposes of the present disclosure is further defined as the percent weight ratio of the crosslinking agent to HA-monomeric units within the crosslinked portion of the HA based composition. It is measured by the weight ratio of crosslinker to HA monomers. In embodiments, the HA has a degree of crosslinking of about 4%, about 5%, about 6%, about 7%, about 8%, about 9%, or about 10%. In some embodiments the HA has a degree of crosslinking of about 6.5%. In other embodiments, the HA has a degree of crosslinking of about 7.5%, or about 8.5%, or about 9.5%, or about 10.5%.

Additionally, the soft-tissue filler compositions may comprise an uncrosslinked HA component greater than about 10% by volume of the filler composition. “Uncrosslinked HA” as used herein refers to individual HA polymer molecules that are not crosslinked. Uncrosslinked HA generally remains water soluble. An uncrosslinked HA fraction may optionally also be included in the composition, for example, to act as a lubricant and facilitate injection into the facial tissues. Such a composition may comprise an uncrosslinked HA fraction where the added uncrosslinked HA is present at a concentration between about 0.1 mg/ml and about 3 mg/ml. Preferably, the uncrosslinked HA may be present at a concentration between about 0.2 mg/ml and about 1.5 mg/ml.

Moreover, the soft-tissue filler compositions may comprise an HA concentration of about 1 mg/ml to about 50 mg/ml. The filler composition may comprise an HA concentration equal to or greater than about 20 mg/ml. In some embodiments, the HA concentration is about 21 mg/ml, or about 22 mg/ml, or about 23 mg/ml, or about 24 mg/ml, or about 25 mg/ml, or about 26 mg/ml, or about 27 mg/ml, or about 28 mg/ml, or about 29 mg/ml, or about 30 mg/ml or greater. In other embodiments, the composition has an HA concentration of between 22.5 mg/ml to 27.5 mg/ml, for example, 25.0 mg/ml.

The soft-tissue filler compositions may also be made with a mixture of low molecular weight hyaluronic acid and high molecular weight hyaluronic acid. As used herein, “molecular weight” means the weight average molecular weight (Mw) in Daltons. As used herein, high molecular weight HA has a molecular weight greater than about 2,000,000 Da (or an intrinsic viscosity greater than 2.2 L/g) and low molecular weight HA has a molecular weight of less than about 1,000,000 Da (or an intrinsic viscosity lower than 1.4 L/g).

Further, the soft-tissue filler compositions may comprise crosslinked hyaluronic acid comprising from about 50% and about 100% of a low molecular weight hyaluronic acid prior to being crosslinked with the crosslinking agent. In some embodiments, the crosslinked hyaluronic acid is made from about 70% to about 90% of a low molecular weight hyaluronic acid prior to being crosslinked with the crosslinking agent. In some embodiments, the crosslinked hyaluronic acid is made from about 90% of a low molecular weight hyaluronic acid prior to being crosslinked with the crosslinking agent.

Additionally, the soft-tissue filler compositions may comprise an anesthetic agent, such as, for example, lidocaine combined with the crosslinked HA component. In embodiments with anesthetics, the concentration of one or more anesthetics is in an amount effective to mitigate pain experienced upon injection of the composition. The at least one local anesthetic can be selected from the group of ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dicyclomine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and salts thereof.

In certain embodiments, the at least one anesthetic agent is lidocaine, such as in the form of lidocaine HCl. The soft-tissue filler compositions of the disclosure may have a lidocaine concentration of between about 0.05% and about 5% by weight of the composition, for example, about 0.05% to about 1.0% by weight of the composition. In some embodiment, the composition has a lidocaine concentration of about 0.3% by weight (w/w %) of the composition. The concentration of lidocaine in the compositions described herein can be therapeutically effective meaning the concentration is adequate to provide a therapeutic benefit without inflicting harm to the patient.

The soft tissue filler composition of the disclosure may be administered subcutaneously and/or supraperiosteally into at least one treatment area of the face of the subject such as the pogonion, the menton, the left pre-jowl sulcus, the right pre-jowl sulcus, and/or the sublabial (mental) crease. In some embodiments, the methods may comprise the step of administering into a treatment area of the patient, an effective amount, for example, about 1.0 ml, or more, for example, about 2.0 ml or more, for example, about 3.0 ml or more, for example, 4.0 ml, of the soft tissue filler composition. The treatment area may be a chin, for example, a retruded chin of a patient. The treatment may comprise administering the composition into two or more of the treatment areas including, over one or more treatment sessions.

The step of administering the soft tissue filler composition to a subject may result in a change in the degree of chin retrusion in the subject. Accordingly, in certain embodiments, a subject categorized as having, for example, severe chin retrusion is treated by administering a soft tissue filler to a treatment area, thereby changing the degree of chin retrusion in the subject. For example, a subject prior to treatment categorized as having severe chin retrusion (i.e., categorized as a 4 on the chin retrusion scale disclosure herein) is categorized following treatment as having moderate-severe (3), moderate (2), minimal (1), or no chin retrusion (0). Alternatively, a subject prior to treatment categorized as having moderate-severe chin retrusion is categorized following treatment as having moderate, minimal, or no chin retrusion. In another example, a subject prior to treatment categorized as having moderate chin retrusion is categorized following treatment as having minimal or no chin retrusion.

The present disclosure also provides methods of determining a change in degree of chin retrusion in a subject. Such methods may comprise the steps of: generating a first image of a face of the subject having a defined chin midpoint, a supramentale line, and a cheilion vertical line; determining from the first image an first position of the defined chin midpoint relative to the supramentale line and the cheilion vertical line; assigning a first degree of chin retrusion in the subject based on the first position of the defined chin midpoint relative to the supramentale line and cheilion vertical line; generating a second image of the face of the subject; determining from the second image a second position of the defined chin midpoint relative to the supramentale line and the cheilion vertical line; and assigning a second degree of chin retrusion in the subject base on the second position of the defined chin midpoint relative to the supramentale line and cheilion vertical line.

A change in the degree of chin retrusion in the subject may be determined after treatment, for example, with a soft tissue filler composition. Some embodiments may further comprise a step of treating the subject before the step of generating a second image. In other embodiments, a change is determined over time, for example as a subject ages.

The present disclosure also provides methods of determining the efficacy of treatment by monitoring the degree of chin retrusion at various times, for example at various times after treatment with a dermal filler. A change in the degree of chin retrusion may be determined at about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, or more, after treatment.

Also provided herein is a computing platform for use by medical providers who treat patients seeking cosmetic procedures. The platform may store and analyze a plurality of images of faces (e.g., several hundred or thousand faces), or information associated with images of faces, and uses machine learning and pattern recognition to categorize a subject with chin retrusion and optionally make treatment recommendations.

Computer systems are also provided having one or more processors and memory storing one or more programs for execution by the one or more processors. Such a system includes memory storing instructions for causing the computer system to perform any of the methods described herein including a portion of any of the methods disclosed herein. For example, the computer system may compare, using a pattern recognition process, one or more aspects of an input image of a face having a defined chin midpoint, a supramentale line, and a cheilion vertical line to corresponding aspects of a plurality of reference images having a defined chin midpoint, a supramentale line, and a cheilion vertical line with associated categories of chin retrusion; obtaining, based on a result of the comparing, a categorization for a degree of chin retrusion for the input image; and optionally recommending a treatment plan based on the categorization of the chin retrusion.

### Illustration of Subject Technology as Clauses

Various examples of aspects of the disclosure are described as numbered clauses (1, 2, 3, etc.) for convenience. These are provided as examples, and do not limit the subject technology.

Clause 1. A method of treating chin retrusion in a subject in need thereof, the method comprising: generating an image of a face of the subject having a defined chin midpoint, a supramentale line, and a cheilion vertical line; determining a position of the defined chin midpoint relative to the supramentale line and the cheilion vertical line; and treating chin retrusion in the subject if the defined chin midpoint is (i) at the supramentale line, (ii) between the supramentale line and the cheilion vertical line, or (iii) at or behind the cheilion vertical line.

Clause 2. The method of Clause 1, wherein the subject is non-Caucasian.

Clause 3. The method of Clause 2, wherein the subject is East Asian.

Clause 4. The method of any one of the preceding Clauses, wherein chin retrusion is treated if the defined chin midpoint is at or behind the cheilion vertical line.

Clause 5. The method of any one of the preceding Clauses, wherein treating the chin retrusion comprises administering a soft tissue filler composition comprising a hyaluronic acid (HA) component crosslinked with a crosslinking agent to the subject.

Clause 6. The method of Clause 5, wherein the crosslinking agent is 1,4-butanediol diglycidyl ether (or 1,4-bis(2,3-epoxypropoxy)butane, 1,2-bis(2,3-epoxypropoxy)ethylene, or 1-(2,3-epoxypropyl)-2,3-epoxycyclohexane.

Clause 7. The method of Clause 5, wherein the HA concentration of the composition is greater than 20 mg/ml.

Clause 8. The method of Clause 5, wherein the HA used for crosslinking comprises high molecular weight HA and low molecular weight HA.

Clause 9. The method of Clause 8, wherein the HA used for crosslinking is a mixture comprising at least 50% by weight low molecular weight HA, based on a total weight of the HA comprising the composition.

Clause 10. The method of Clause 5, wherein the HA has a degree of crosslinking of between about 4% and about 10%.

Clause 11. The method of Clause 10, wherein the HA has a degree of crosslinking of about 4%.

Clause 12. The method of Clause 10, wherein the HA has a degree of crosslinking of about 6%.

Clause 13. The method of Clause 10, wherein the HA has a degree of crosslinking of about 6.5%.

Clause 14. The method of Clause 10, wherein the HA has a degree of crosslinking of about 8%.

Clause 15. The method of Clause 10, wherein the HA has a degree of crosslinking of about 10%.

Clause 16. The method of Clause 5, wherein the soft tissue filler composition comprises an uncrosslinked HA component greater than about 10% by volume of the filler composition.

Clause 17. The method of Clause 5, wherein the soft tissue filler composition further comprises lidocaine combined with the crosslinked HA component.

Clause 18. The method of Clause 17, wherein the lidocaine is present at a concentration of about 0.05% w/w to about 1% w/w.

Clause 19. The method of Clause 5, wherein the soft tissue filler composition is administered subcutaneously and/or supraperiosteally into at least one treatment area of the face of the subject, and wherein the treatment area is selected from one or more of the pogonion, the mentum, the left pre jowl sulcus, the right pre jowl sulcus, and the sublabial (mental) crease.

Clause 20. A method of categorizing a subject based on a degree of chin retrusion comprising: generating an image of a face of the subject having a defined chin midpoint, a labrale inferius line, a supramentale line, and a cheilion vertical line; determining a position of the defined chin midpoint relative to one or more of the labrale inferius line, the supramentale line, and/or the cheilion vertical line; and categorizing a degree of chin retrusion in the subject based on the position of the defined chin midpoint relative to the labrale inferius line, the supramentale line, and/or cheilion vertical line.

Clause 21. The method of Clause 20, wherein the subject is categorized as having no chin retrusion if the defined chin midpoint is at or in front on the labrale inferius line.

Clause 22. The method of Clause 21, wherein the subject is categorized as having minimal chin retrusion if the defined chin midpoint is between the labrale inferius line and the supramentale line.

Clause 23. The method of any one of Clauses 21 to 22, wherein the subject is categorized as having moderate chin retrusion if the defined chin midpoint is at the supramentale line.

Clause 24. The method of any one of Clauses 21 to 23, wherein the subject is categorized as having moderate-severe chin retrusion if the defined chin midpoint is between the supramentale line and the cheilion vertical line.

Clause 25. The method of any one of Clauses 21 to 24, wherein the subject is categorized as having severe chin retrusion if the defined chin midpoint is at or behind the cheilion vertical line.

Clause 26. A chin retrusion scale comprising a plurality of facial images representing a plurality of grades of chin retrusion, wherein each image comprises a profile view of a face showing at least a defined chin midpoint and one or more of a labrale inferius line, a supramentale line, and a cheilion line, wherein at least one of the grades of chin retrusion is based on a relative position of the defined chin midpoint and the labrale inferius line, the supramentale line, and/or the cheilion line.

Clause 27. The chin retrusion scale of Clause 26, wherein the scale is a photonumeric scale.

Clause 28. The chin retrusion scale of Clause 27, wherein the photonumeric scale comprises scores 0-4, and wherein: a score of 0 signifies no chin retrusion in a subject with a defined chin midpoint at or in front on the labrale inferius line; a score of 1 signifies minimal chin retrusion in a subject with a defined chin midpoint between the labrale inferius line and the supramentale line; a score of 2 signifies moderate chin retrusion in a subject with a defined chin midpoint at the supramentale line; a score of 3 signifies moderate-severe chin retrusion in a subject with a defined chin midpoint between the supramentale line and the cheilion vertical line; and a score of 4 signifies severe chin retrusion in a subject with a defined chin midpoint at or behind the cheilion vertical line.

Clause 29. A method of treating chin retrusion in a subject in need thereof comprising: generating an image of the face of a subject having a defined chin midpoint, and one or more of a supramentale line and a cheilion vertical line; and categorizing a degree of chin retrusion in the subject, wherein moderate chin retrusion is present if the defined chin midpoint is at the supramentale line, wherein severe chin retrusion is present if the defined chin midpoint is between the supramentale line and the cheilion vertical line, wherein severe chin retrusion is present if the defined chin midpoint is at or behind the cheilion vertical line, and wherein the subject is administered a treatment if categorized as having moderate chin retrusion, moderate-severe chin retrusion, or severe chin retrusion.

Clause 30. The method of Clause 29, wherein the treatment is a soft tissue filler composition comprising a hyaluronic acid (HA) component crosslinked with a crosslinking agent.

Clause 31. The method of Clause 30, wherein the crosslinking agent is 1,4-butanediol diglycidyl ether (or 1,4-bis(2,3-epoxypropoxy)butane, 1,2-bis(2,3-epoxypropoxy)ethylene, or 1-(2,3-epoxypropyl)-2,3-epoxycyclohexane.

Clause 32. The method of any one of Clauses 30 to 31, wherein the soft tissue filler composition comprises an uncrosslinked HA component greater than about 10% by volume of the filler composition.

Clause 33. The method of any one of Clauses 30 to 32, wherein the soft tissue filler composition further comprises lidocaine combined with the crosslinked HA component.

Clause 34. The method of Clause 33, wherein the lidocaine is present at a concentration of about 0.05% w/w to about 1% w/w.

Clause 35. A method of treating chin retrusion in a subject in need thereof comprising: generating an image of the face of a subject comprising a defined chin midpoint, a supramentale line, and a cheilion vertical line; and treating chin retrusion in the subject if the defined chin midpoint is between the supramentale line and the cheilion vertical line, or at or behind the cheilion vertical line, wherein treating the subject comprises supraperiostally administering into at least one treatment area of the face of the patient, an effective amount of a composition comprising BDDE-crosslinked hyaluronic acid (HA), the HA having a degree of crosslinking of about 6.5%, and having a HA concentration of greater than 20 mg/ml, wherein the HA used for crosslinking is a mixture containing at least 50% by weight of low molecular weight HA, based on a total weight of the HA; the treatment area being selected from one or more of the pogonion, the mentum, the left pre-jowl sulcus, the right pre-jowl sulcus, and the sublabial crease.

Clause 36. A method for determining a change in degree of chin retrusion in a subject, the method comprising: generating a first image of a face of the subject having a defined chin midpoint, a supramentale line, and a cheilion vertical line; determining from the initial image, an initial position of the defined chin midpoint relative to the supramentale line and the cheilion vertical line; and assigning a first degree of chin retrusion in the subject based on the first position of the defined chin midpoint relative to the supramentale line and cheilion vertical line; generating a second image of the face of the subject; determining from the second image, a second position of the defined chin midpoint relative to the supramentale line and the cheilion vertical line; and assigning a second degree of chin retrusion in the subject based on the subsequent position of the defined chin midpoint relative to the supramentale line and cheilion vertical line.

Clause 37. The method of Clause 36, further comprising administering a treatment to a treatment area on the face of the subject before the step of generating a second image.

Clause 38. The method of Clause 37, wherein the treatment comprises a soft tissue filler composition.

Clause 39. The method of any one of Clauses 36 to 38, wherein the second degree of chin retrusion is less than the first degree of chin retrusion.

Clause 40. The method of any one of Clauses 36 to 39, wherein the second image is generated at between about 1 month and about 24 months after the first image.

Clause 41. An electronic computer system, comprising: one or more processors; and memory storing one or more programs for execution by the one or more processors, the one or more programs comprising instructions for: obtaining an input image of a face of a subject having a defined chin midpoint and one or more of a labrale inferius line, a supramentale line, and/or a cheilion vertical line; comparing, using a pattern recognition process, one or more aspects of the input image to corresponding aspects of a plurality of reference images of faces each having a defined chin midpoint and one or more of a labrale inferius line, a supramentale line, and/or a cheilion vertical line; categorizing, based on a result of the comparing, a degree of chin retrusion in the subject; and recommending a treatment plan based on the categorization of the subject, wherein the reference images each have a scored degree of chin retrusion.

Clause 42. The electronic computer system of Clause 41, wherein the treatment plan comprises supraperiostally administering into at least one treatment area of the face of the patient, an effective amount of a composition comprising BDDE-crosslinked hyaluronic acid (HA), the treatment area being selected from one or more of the pogonion, the mentum, the left pre-jowl sulcus, the right pre jowl sulcus, and the sublabial crease.

Clause 43. The electronic computer system of any one of Clauses 41 to 42, wherein the one or more aspects of the input image correspond with physical features of the face that are detectable in the input image.

### EXAMPLES

### Methods

### Example 1: Validation of Chin Retrusion Scale

Scale Validation Process. A chin retrusion scale according to the disclosure has 5 categories, each of which is associated with a numeric score and a descriptive grade (Table 1). Numeric scores range from 0 to 4, with 0 representing no retrusion and 4 representing severe retrusion. The chin retrusion scale contains morphed subject photographs for all 5 severities of chin retrusion; the base image is a photograph of a subject with grade 2 retrusion. In addition, the scale contains real example images for all 5 severities including both sexes. An exemplary embodiment of the scale is shown in FIG. 1.

TABLE 1
Descriptors for a Chin Retrusion Scale

| Score | Grade | Descriptor |
|---|---|---|
| 0 | None | No chin retrusion; defined chin midpoint at or |
| | | in front of the labrale inferius line |
| 1 | Minimal | Minimal chin retrusion; defined chin midpoint |
| | | is between the supramentale line and the labrale |
| | | inferius line |
| 2 | Moderate | Moderate chin retrusion; defined chin midpoint |
| | | at the supramentale line |
| 3 | Moderate-Severe | Moderate-Severe chin retrusion; defined chin |
| | | midpoint between the supramentale line and the |
| | | cheilion vertical line |
| 4 | Severe | Severe chin retrusion; defined chin midpoint at |
| | | or behind the cheilion vertical line |

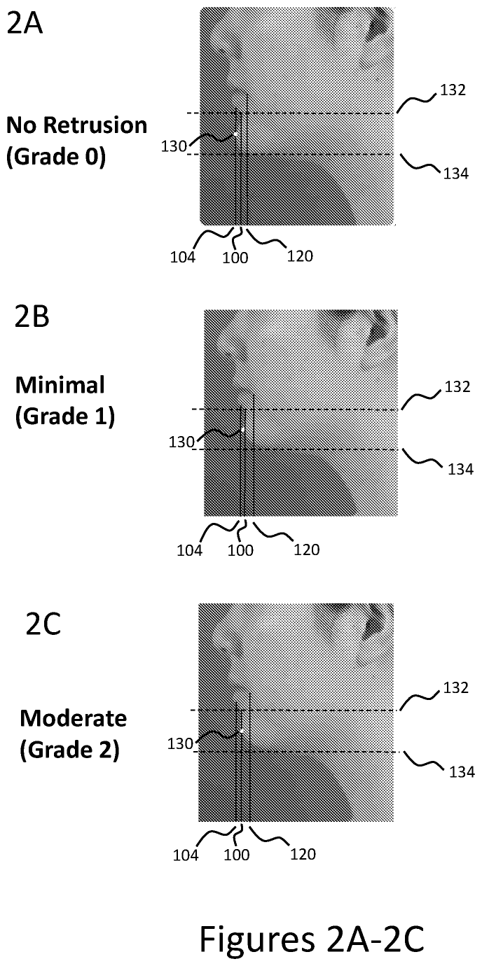

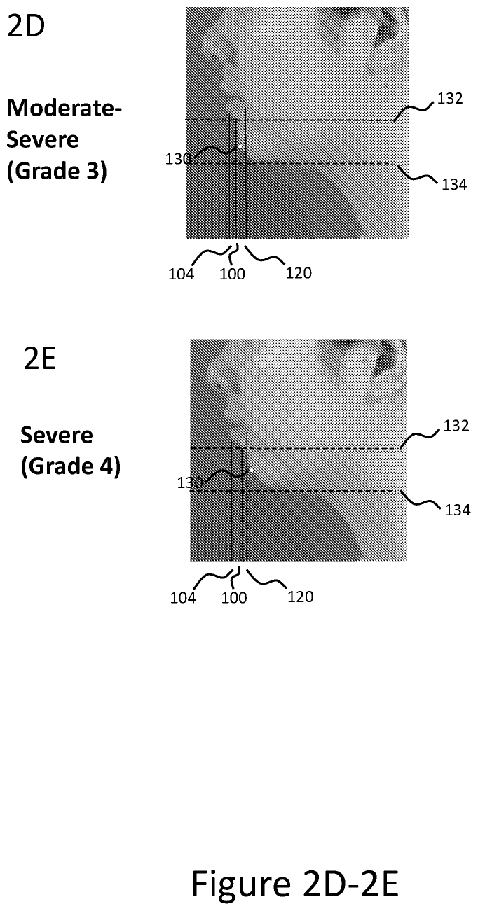

FIG. 2 presents the lateral view of the anatomic markers of the lower face used to assess chin retrusion. Using this assessment guide, chin retrusion is judged by the location of the defined chin midpoint in relation to the cheilion, labrale inferius, and supramentale.

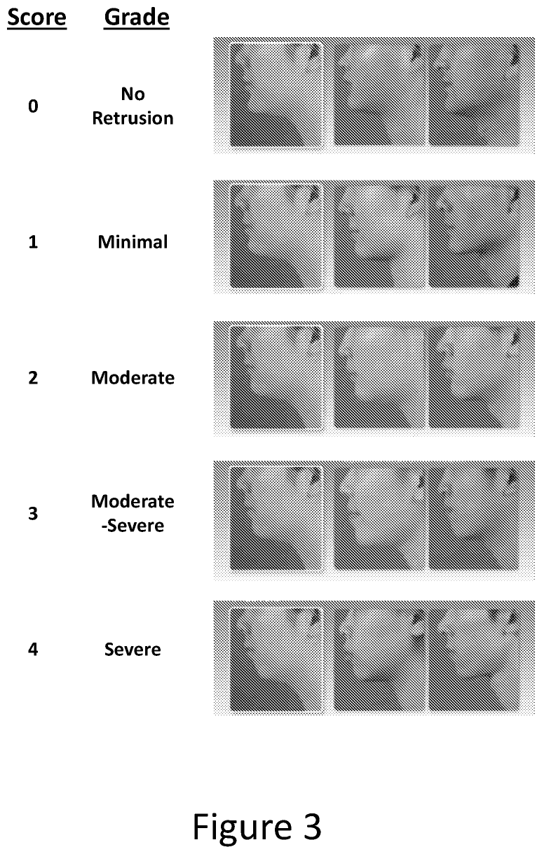

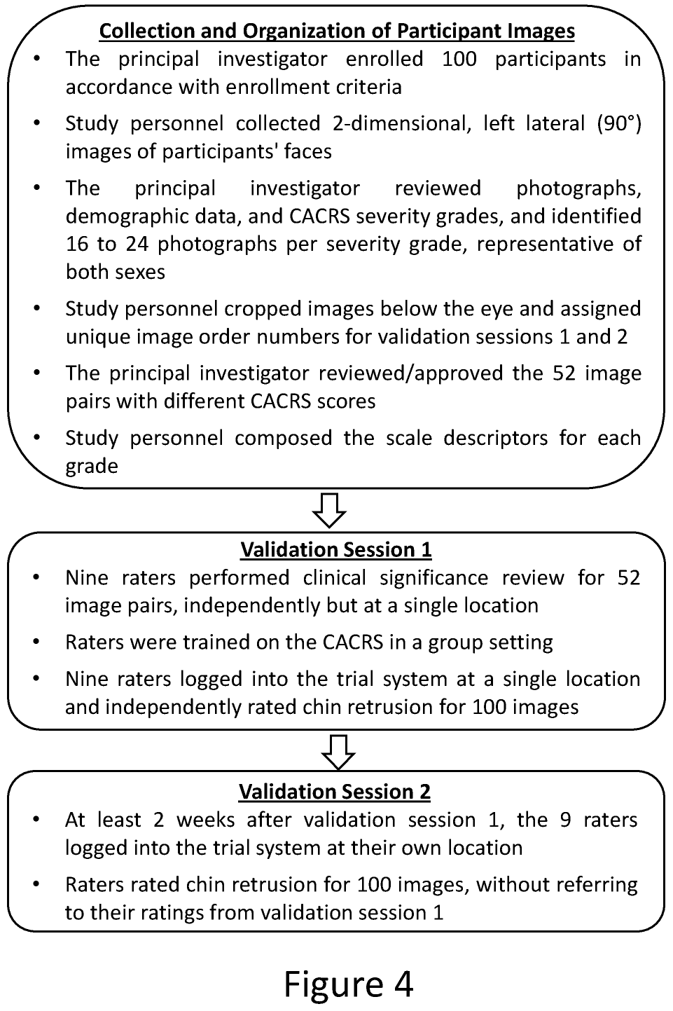

To validate an exemplary chin retrusion scale, a team of 10 physicians based in China, consisting of 1 principal investigator and 9 independent raters experienced in facial aesthetics, assessed a pool of subject images using image rating and electronic data capture tools provided by Canfield Scientific, Inc. (Canfield, Fairfield, N.J.). FIG. 3 depicts the key steps in the process of validating an embodiment of the chin retrusion scale of the disclosure. Two effectiveness measures were used for scale validation: rater evaluations of clinically significant differences in select image pairs and rater assessment of chin retrusion using the chin retrusion scale of the disclosure. This study was conducted in conformance with the principles of the Declaration of Helsinki or the laws and regulations of the country in which the research was conducted, whichever afforded greater protection for the individual.

Subjects. A total of 300 subjects were enrolled to have their photographs taken, with images of 100 subjects planned for inclusion in the assessment. The subjects were required to be men and women of Han nationality from Beijing aged 18 to 50 years, in good general health, with varying degrees of chin retrusion. All subjects provided written informed consent. Subjects were excluded if they appeared in training images for the chin retrusion scale, had substantial congenital or traumatic cephalofacial deformity, scarring, or defects that interfere with visual assessment, or had a history of cephalofacial surgery.

Subject Images and Validation Sessions. Using standardized equipment to capture 2-dimensional images (i.e., Canon PowerShot G16, 12.1 MP camera), subjects were photographed, without smiling, from a left, lateral, 90° view in natural light, with a relaxed, natural posture and with tragus points exposed and the Frankfort horizontal plane parallel to the floor.

Study personnel developed 2 randomization schemes for subject images to be used in 2 validation sessions that occurred at least 2 weeks apart. For validation session 1, 52 pairs of images were selected for the clinical significance assessment. The principal investigator reviewed and approved the image pairs. Image pairs represented 1-, 2-, 3-, and 4-grade differences in retrusion scores on the chin retrusion scale and represented all levels of difference among chin retrusion scale grades. Validation session 1 took place in a group setting at a single location, and the clinical significance assessment was conducted before raters received training on the chin retrusion scale. For each of the 52 pairs of images assembled by the principal investigator, raters were asked to designate the images as either clinically significantly different or not different.

The raters then received training on how to assess chin retrusion using a chin retrusion scale of the disclosure and participated in group discussion and consensus building. The raters next viewed the 100 images of the Chinese subjects, and independently graded the category of chin retrusion for each subject.

Validation session 2 took place in an individual setting at the rater's own location. Each rater logged into a secure website, viewed the 100 images of the Chinese subjects, and graded the category of chin retrusion for each subject.

Data Analysis and Statistics. Sample size was calculated using the method described by Bonett et al. (“Sample size requirements for estimating intraclass correlations with desired precision,” Stat Med. 2002; 21(9):1331-1335). All statistical analyses were performed using SAS version 9.2 or higher (Cary, N.C., USA). With at least 8 raters and an intraclass correlation coefficient (ICC) of 0.60, a total of 100 subjects provided 95% CIs with a width of at least 0.16 for interrater reliability. A minimum of 16 subject images and a maximum of 24 subject images were selected for each category of the chin retrusion scale.

The SAS macro “INTRACC” was used to determine interrater reliability by calculating the ICC and 95% CI for validations sessions 1 and 2. (Shrout P E, Fleiss J L, Intraclass correlations: uses in assessing rater reliability,” Psychol Bull. 1979; 86(2):420-428.) For each rater, the SAS procedure “FREQ” was used to determine intrarater reliability by calculating a weighted kappa with Cicchetti-Allison weights (SAS default weights) and 95% CI. (Cicchetti D V, Allison T, “A new procedure for assessing reliability of scoring EEG sleep recordings,” Amer J EEG Tech. 1971; 11(3):101-110.) The overall mean weighted kappa was computed by averaging the overall weighted kappa for each rater from validation sessions 1 and 2.

Agreement was evaluated as defined by Landis and Koch. (Landis J R, Koch G G, “The measurement of observer agreement for categorical data,” Biometrics. 1977; 33(1):159-174.) Five degrees of agreement were characterized: slight (0.00-0.20), fair (0.21-0.40), moderate (0.41-0.60), substantial (0.61-0.80), and almost perfect (0.81-1.00).

To assess the clinical significance of paired images, the percentage of image pairs identified as clinically significantly different, or not different, were presented for all absolute differences in chin retrusion scores.

### Results

Subject Demographics. Nearly two-thirds of the population (62%) was female. The mean (SD) age of the subjects was 27.2 (7.8) years.

Scale Validation. The chin retrusion scale of the disclosure demonstrated almost perfect interrater agreement during sessions 1 and 2, with ICCs of 0.89 and 0.90, respectively (Table 2). Overall intrarater agreement for all 9 raters was almost perfect, with a weighted kappa of 0.82 (Table 2). Chin retrusion scale agreement was almost perfect for 6 of 9 raters and substantial for the remaining 3 raters. The difference in weighted kappas between the highest- and lowest-performing raters was 0.21.

TABLE 2
Estimates of Agreement for the Chin Retrusion Scale

| | |
|---|---|
| Interrater agreement, ICC (95% CI) | |
| Validation session 1 | 0.89 (0.86-0.92) |
| Validation session 2 | 0.90 (0.86-0.92) |
| Intrarater agreement | |
| Mean weighted kappa (95% CI) | 0.82 (0.80-0.84) |
| ICC, intraclass correlation coefficient. | |

Clinical Significance. The 9 raters evaluated all 52 image pairs, for a total of 468 assessments (Table 3). In total, 154, 210, 64, 27, and 13 assessments represented pairs with no difference in CACRS category score and pairs with an absolute difference of 0, 1, 2, 3, and 4, respectively. Additionally, 67% of pairs of images with an absolute difference score of 1 and 56% of pairs of images with an absolute difference score of 0 had a clinically significant difference rating (Table 3). The percentages of raters who identified clinically significant differences between images increased with the absolute difference between pairs (92.2% for image pairs with an absolute difference score of 2, and 100% for pairs with an absolute difference of 3 or 4; Table 3). Based on these data, a 1-grade difference was determined to be clinically significant.

TABLE 3
Clinical Significance of Rating Pair Differences

| Scores* | Clinical Difference | % (n) | |
|---|---|---|---|
| 0 | Yes | 55.8 | (86) |
| | No | 44.2 | (68) |
| 1 | Yes | 66.7 | (140) |
| | No | 33.3 | (70) |
| 2 | Yes | 92.2 | (59) |
| | No | 7.8 | (5) |
| 3 | Yes | 100 | (27) |
| | No | 0 | (0) |
| 4 | Yes | 100 | (13) |
| | No | 0 | (0) |

*Absolute difference in score of the 2 images in a pair.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. In addition, it is not necessary for a method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method of treating chin retrusion in a subject in need thereof, the method comprising:
generating an image of a face of the subject having a defined chin midpoint, a supramentale line, and a cheilion vertical line;
determining a position of the defined chin midpoint relative to the supramentale line and the cheilion vertical line; and
treating chin retrusion in the subject if the defined chin midpoint is (i) at the supramentale line, (ii) between the supramentale line and the cheilion vertical line or (iii) at or behind the cheilion vertical line.

2. The method of claim 1, wherein the subject is non-Caucasian.

3. The method of claim 2, wherein the subject is East Asian.

4. The method of claim 1, wherein chin retrusion is treated if the defined chin midpoint is at or behind the cheilion vertical line.

5. The method of claim 1, wherein treating chin retrusion comprises administering a soft tissue filler composition comprising a hyaluronic acid (HA) component crosslinked with a crosslinking agent to the subject.

6. The method of claim 5, wherein the crosslinking agent is 1,4-butanediol diglycidyl ether (or 1,4-bis(2,3-epoxypropoxy)butane, 1,2-bis(2,3-epoxypropoxy)ethylene, or 1-(2,3-epoxypropyl)-2,3-epoxycyclohexane.

7. The method of claim 5, wherein the HA concentration of the composition is greater than 20 mg/ml.

8. The method of claim 5, wherein the HA used for crosslinking comprises high molecular weight HA and low molecular weight HA.

9. The method of claim 8, wherein the HA used for crosslinking is a mixture comprising at least 50% by weight low molecular weight HA, based on a total weight of the HA comprising the composition.

10. The method of claim 5, wherein the HA has a degree of crosslinking of between about 4% and about 10%.

11. The method of claim 10, wherein the HA has a degree of crosslinking of about 4%.

12. The method of claim 10, wherein the HA has a degree of crosslinking of about 6%.

13. The method of claim 10, wherein the HA has a degree of crosslinking of about 6.5%.

14. The method of claim 10, wherein the HA has a degree of crosslinking of about 8%.

15. The method of claim 10, wherein the HA has a degree of crosslinking of about 10%.

16. The method of claim 5, wherein the soft tissue filler composition comprises an uncrosslinked HA component greater than about 10% by volume of the filler composition.

17. The method of claim 5, wherein the soft tissue filler composition further comprises lidocaine combined with the crosslinked HA component.

18. The method of claim 17, wherein the lidocaine is present at a concentration of about 0.05% w/w to about 1% w/w.

19. The method of claim 5, wherein the soft tissue filler composition is administered subcutaneously and/or supraperiosteally into at least one treatment area of the face of the subject, wherein the treatment area is selected from the group consisting of the pogonion, the mentum, the left pre-jowl sulcus, the right pre-jowl sulcus, and the sublabial (mental) crease.

20. A method of categorizing a subject based on a degree of chin retrusion comprising:
generating an image of a face of the subject having a defined chin midpoint, and two or more of a labrale inferius line, a supramentale line, and a cheilion vertical line;
determining a position of the defined chin midpoint relative to the labrale inferius line, the supramentale line, and/or cheilion vertical line; and
categorizing a degree of chin retrusion in the subject based on the position of the defined chin midpoint relative to the labrale inferius line, the supramentale line and/or cheilion vertical line.

21. The method of claim 20, wherein the subject is categorized as having no chin retrusion if the defined chin midpoint is at or in front of the labrale inferius line.

22. The method of claim 21, wherein the subject is categorized as having minimal chin retrusion if the defined chin midpoint is between the labrale inferius line and the supramentale line.

23. The method of claim 21, wherein the subject is categorized as having moderate chin retrusion if the defined chin midpoint is at the supramentale line.

24. The method of claim 21, wherein the subject is categorized as having moderate-severe chin retrusion if the defined chin midpoint is between the supramentale line and the cheilion vertical line.

25. The method of claim 21, wherein the subject is categorized as having severe chin retrusion if the defined chin midpoint is at or behind the cheilion vertical line.

26. A chin retrusion scale comprising a plurality of facial images representing a plurality of grades of chin retrusion, wherein each image comprises a profile view of a face showing a defined chin midpoint and at least one of a labrale inferius line, a supramentale line, and a cheilion line, and wherein at least one of the grades of chin retrusion is based on a relative position of the chin midpoint and the labrale inferius line, the supramentale line, or the cheilion line.

27. The chin retrusion scale of claim 26, wherein the scale is a photonumeric scale.

28. The chin retrusion scale of claim 27, wherein the photonumeric scale comprises scores 0-4, and wherein:
a score of 0 signifies no chin retrusion in a subject with a defined chin midpoint at or in front on the labrale inferius line;
a score of 1 signifies minimal chin retrusion in a subject with a defined chin midpoint between the labrale inferius line and the supramentale line;
a score of 2 signifies moderate chin retrusion in a subject with a defined chin midpoint at the supramentale line;
a score of 3 signifies moderate-severe chin retrusion in a subject with a defined chin midpoint between the supramentale line and the cheilion vertical line; and
a score of 4 signifies severe chin retrusion in a subject with a defined chin midpoint at or behind the cheilion vertical line.

29. A method of treating chin retrusion in a subject in need thereof comprising:
generating an image of the face of a subject having a defined chin midpoint and one or more of a supramentale line and a cheilion vertical line; and
categorizing a degree of chin retrusion in the subject,
wherein moderate chin retrusion is present if the defined chin midpoint is at the supramentale line,
wherein moderate-severe chin retrusion is present if the defined chin midpoint is between the supramentale line and the cheilion vertical line;
wherein severe chin retrusion is present if the defined chin midpoint is at or behind the cheilion vertical line; and
wherein the subject is administered a treatment if categorized as having moderate, moderate-severe, or severe chin retrusion.

30. The method of claim 29, wherein the treatment comprises a soft tissue filler composition comprising a hyaluronic acid (HA) component crosslinked with a crosslinking agent.

31. The method of claim 30, wherein the crosslinking agent is 1,4-butanediol diglycidyl ether (or 1,4-bis(2,3-epoxypropoxy)butane, 1,2-bis(2,3-epoxypropoxy)ethylene, or 1-(2,3-epoxypropyl)-2,3-epoxycyclohexane.

32. The method of claim 30, wherein the soft tissue filler composition comprises an uncrosslinked HA component greater than about 10% by volume of the filler composition.

33. The method of claim 30, wherein the soft tissue filler composition further comprises lidocaine combined with the crosslinked HA component.

34. The method of claim 33, wherein the lidocaine is present at a concentration of about 0.05% w/w to about 1% w/w.

35. A method of treating chin retrusion in a subject in need thereof comprising:
generating an image of the face of a subject comprising a defined chin midpoint and one or more of a supramentale line and a cheilion vertical line; and
treating chin retrusion in the subject if the defined chin midpoint is at the supramentale line, between the supramentale line and the cheilion vertical line, or at or behind the cheilion vertical line,
wherein treating the subject comprises supraperiostally administering into at least one treatment area of the face of the patient, an effective amount of a composition comprising BDDE-crosslinked hyaluronic acid (HA), the HA having a degree of crosslinking of about 6.5%, and having a HA concentration of greater than 20 mg/ml, wherein the HA used for crosslinking is a mixture containing at least 50% by weight of low molecular weight HA, based on a total weight of the HA; the treatment area being selected from the group consisting of the pogonion, the mentum, the left pre-jowl sulcus, the right pre-jowl sulcus, and the sublabial crease.

36. A method for determining a change in degree of chin retrusion in a subject, the method comprising:
generating a first image of a face of the subject having a defined chin midpoint, and at least one of a labrale inferius line, a supramentale line, and a cheilion vertical line;
determining from the initial image, an initial position of the defined chin midpoint relative to the labrale inferius line, supramentale line, and/or the cheilion vertical line;
assigning a first degree of chin retrusion in the subject based on the first position of the defined chin midpoint relative to the labrale inferius line, the supramentale line, and/or cheilion vertical line;
generating a second image of the face of the subject;
determining from the second image, a second position of the defined chin midpoint relative to the labrale inferius line, the supramentale line, and/or the cheilion vertical line; and
assigning a second degree of chin retrusion in the subject based on the subsequent position of the defined chin midpoint relative to the labrale inferius line, the supramentale line, and the cheilion vertical line.

37. The method of claim 36, further comprising administering a treatment to a treatment area on the face of the subject before the step of generating a second image.

38. The method of claim 37, wherein the treatment comprises a soft tissue filler composition.

39. The method of claim 36, wherein the second degree of chin retrusion is less than the first degree of chin retrusion.

40. The method of claim 36, wherein the second image is generated at between about 1 month and about 24 months after the first image.

41. An electronic computer system, comprising:
one or more processors; and
memory storing one or more programs for execution by the one or more processors, the one or more programs comprising instructions for:
obtaining an input image of a face of a subject having a defined chin midpoint, and one or more of a labrale inferius line, a supramentale line, and a cheilion vertical line;
comparing, using a pattern recognition process, one or more aspects of the input image to corresponding aspects of a plurality of reference images of faces each having a defined chin midpoint, a labrale inferius line, a supramentale line, and a cheilion vertical line;
categorizing, based on a result of the comparing, a degree of chin retrusion in the subject; and
recommending a treatment plan based on the categorization of the subject, wherein the reference images each have a scored degree of chin retrusion.

42. The electronic computer system of claim 41, wherein the treatment plan comprises supraperiostally administering into at least one treatment area of the face of the patient, an effective amount of a composition comprising BDDE-crosslinked hyaluronic acid (HA), wherein the treatment is one or more of the pogonion, the mentum, the left pre jowl sulcus, the right pre jowl sulcus, and the sublabial crease.

43. The electronic computer system of claim 41, wherein the one or more aspects of the input image correspond with physical features of the face that are detectable in the input image.
